(19) United States
(12) Patent Application Publication
(10) Pub. No.: US 2026/0193444 A1
(43) Pub. Date: Jul. 9, 2026

(54) METHOD FOR DEPOLYMERIZING POLYETHYLENE TEREPHTHALATE

(71) Applicant: KING FAHD UNIVERSITY OF PETROLEUM AND MINERALS, Dhahran (SA)

(72) Inventors: Samir BARMAN, Dhahran (SA); Rajesh THERAVALAPPIL, Dhahran (SA); Niladri MAITY, Dhahran (SA); Jaseer Ettapurath ANTHOLINDAVIDA, Dhahran (SA); Nestor Garcia VILLALTA, Dhahran (SA)

(73) Assignee: KING FAHD UNIVERSITY OF PETROLEUM AND MINERALS, Dhahran (SA)

(21) Appl. No.: 19/012,376

(22) Filed: Jan. 7, 2025

(51) Int. Cl. C08J 11/26; C07C 67/297; C07C 67/60; C08J 11/24; C08J 11/28

(52) U.S. Cl. CPC C08J 11/26; C07C 67/297; C07C 67/60; C08J 11/24; C08J 11/28; C08J 2367/06

(57) ABSTRACT

A method of depolymerizing polyethylene terephthalate that includes heating to 140 to 210° C. a reaction mixture including polyethylene terephthalate, a salt, urea, and a glycol. The salt includes an alkali or alkaline earth metal cation and a carboxylate having 1 to 6 carbon atoms. The reaction results in depolymerization to form a glycol ester of terephthalic acid including the glycol.

### STATEMENT OF ACKNOWLEDGEMENT

Support from the Center for Refining and Advanced Chemicals at King Fahd University of Petroleum and Minerals under grant INRC2208 is gratefully acknowledged.

### BACKGROUND

### Technical Field

The present disclosure is directed to depolymerization of polyethylene terephthalate (PET).

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Plastics are essential in everyday life due to their functional properties and versatility, but the rising production of over 400 million metric tons annually has resulted in a significant waste accumulation crisis. Some plastics are recyclable. This recyclability, however, has varying degrees of utility. For a few plastics, the recycling involves being able to completely reform a plastic article into a new plastic article. For many plastics, however, the recycling involves little more than mechanical grinding or shredding to form a filler or additive to other materials. There is no way to actually produce a new article from the plastic. This type of limited re-purposing does not restart a lifecycle of the plastic.

A more useful way to recycle certain plastics would be transform them back into the constituent parts. Depolymerizing plastics involves breaking down the polymer into its monomers, enabling reuse in new products by re-polymerizing the regenerated monomers.

Polyethylene terephthalate (PET), widely used in packaging, poses environmental challenges due to its accumulation in landfills and oceans. Depolymerization offers a sustainable solution by chemically recycling PET, unlike mechanical recycling, which degrades plastic quality over time.

Accordingly, it is one object of the present disclosure to provide a method for depolymerizing of polyethylene terephthalate (PET) into constituent monomers by utilizing environmentally friendly catalysts that may circumvent the drawbacks and limitations such as non-biodegradability, toxicity, and limited selectivity of other catalysts.

### SUMMARY

According to a first aspect, the present disclosure relates to a method of depolymerizing polyethylene terephthalate. In some embodiments, the method includes heating to 140 to 210° C. a reaction mixture including polyethylene terephthalate, a salt, urea, and a glycol. In some embodiments, the salt includes an alkali or alkaline earth metal cation and a carboxylate having 1 to 6 carbon atoms. In some embodiments, the depolymerizing forms a glycol ester of terephthalic acid including the glycol.

In some embodiments, the salt includes an alkali or alkaline earth metal cation and a carboxylate having 1 to 6 carbon atoms is at least one selected from the group consisting of lithium acetate, lithium formate, sodium acetate, sodium formate, potassium acetate, potassium formate, calcium acetate, calcium formate, magnesium acetate, and magnesium formate.

In some embodiments, the salt includes an alkali or alkaline earth metal cation and a carboxylate having 1 to 6 carbon atoms is present in an amount of 0.1 to 10 wt. % based on a total weight of polyethylene terephthalate.

In some embodiments, the glycol is ethylene glycol.

In some embodiments, the glycol ester of terephthalic acid includes the glycol is bis(2-hydroxyethyl) terephthalate.

In some embodiments, the reaction mixture further includes an amine base.

In some embodiments, the amine base is at least one selected from the group consisting of 1,8-Diazabicyclo(5.4.0)undec7-ene, 1,5-Diazabicyclo(4.3.0)non-5-ene,1,4-diazabicyclo[2.2.2]octane, 1,5,7-Triazabicyclo[4.4.0]dec-5-ene, N,N-Dimethylacetamide, Imidazole, 1-Methylimidazole, 2-Methylimidazole, Benzimidazole/Triphenylphosphine, 4-(Dimethylamino)pyridine, Triethylamine, Quinuclidine, 7-Methyl-1,5,7-triazabicyclo[4.4.0]dec-5-ene, 1,1,3,3-Tetramethylguanidine, 2-tert-Butylimino-2-diethylamino-1,3-dimethylperhydro-1,3,2-diazaphosphorine.

In some embodiments, the amine base is an amidine base. In some embodiments, the amidine base is at least one selected from the group consisting of 1,8-Diazabicyclo(5.4.0)undec7-ene, 1,5-Diazabicyclo(4.3.0)non-5-ene, 1,5,7-Triazabicyclo[4.4.0]dec-5-ene, 7-Methyl-1,5,7-triazabicyclo[4.4.0]dec-5-ene, and 1,1,3,3-Tetramethylguanidine.

In some embodiments, the amine base is present in an amount of 0.1 to 10 wt. % based on a total weight of polyethylene terephthalate.

In some embodiments, the urea is present in an amount of 0.1 to 10 wt. % based on a total weight of polyethylene terephthalate.

In some embodiments, the reaction mixture has a mole ratio of salt including an alkali or alkaline earth metal cation and a carboxylate having 1 to 6 carbon atoms to urea of 2.5:1 to 1:2.5.

In some embodiments, the method has a polyethylene terephthalate conversion of 32 to 52.5% based on an initial weight of polyethylene terephthalate at a temperature of 140 to 170° C. The method has a selectivity of glycol ester of terephthalic acid of 82.5 to 99.9% at a temperature of 140 to 170° C.

In some embodiments, the method has a polyethylene terephthalate conversion of 72.5 to 100% based on the initial weight of polyethylene terephthalate at a temperature of greater than 170 to 210° C. The method has selectivity of glycol ester of terephthalic acid of 82.5 to 99.9% at a temperature of greater than 170 to 210° C.

In some embodiments, the method reaches a maximum conversion of polyethylene terephthalate in 2 to 4 hours.

In some embodiments, the reaction mixture is substantially free of zinc.

The present disclosure also relates to a method of depolymerizing polyethylene terephthalate. In some embodiments, the method includes heating a reaction mixture to 140 to 210° C. including polyethylene terephthalate, a salt, an amine base, and a glycol. In some embodiments, the salt includes an alkali or alkaline earth metal cation and a carboxylate having 1 to 6 carbon atoms. In some embodiments, the depolymerizing forms a glycol ester of terephthalic acid including the glycol. In some embodiments, the reaction mixture is substantially free of urea.

In some embodiments, the amine base is at least one selected from the group consisting of 1,8-Diazabicyclo(5.4.0)undec7-ene, 1,5-Diazabicyclo(4.3.0)non-5-ene,1,4-diazabicyclo[2.2.2]octane, 1,5,7-Triazabicyclo[4.4.0]dec-5-ene, N,N-Dimethylacetamide, Imidazole, 1-Methylimidazole, 2-Methylimidazole, Benzimidazole/Triphenylphosphine, 4-(Dimethylamino)pyridine, Triethylamine, Quinuclidine, 7-Methyl-1,5,7-triazabicyclo[4.4.0]dec-5-ene, 1,1,3,3-Tetramethylguanidine, 2-tert-Butylimino-2-diethylamino-1,3-dimethylperhydro-1,3,2-diazaphosphorine.

In some embodiments, the amine base is an amidine base. In some embodiments, the amidine base is at least one selected from the group consisting of 1,8-Diazabicyclo(5.4.0)undec7-ene, 1,5-Diazabicyclo(4.3.0)non-5-ene, 1,5,7-Triazabicyclo[4.4.0]dec-5-ene, 7-Methyl-1,5,7-triazabicyclo[4.4.0]dec-5-ene, and 1,1,3,3-Tetramethylguanidine.

In some embodiments, the method has a polyethylene terephthalate conversion of 32 to 52.5% based on the initial weight of polyethylene terephthalate at a temperature of 140 to 170° C. The method has a selectivity of glycol ester of terephthalic acid of 82.5 to 99.9% at a temperature of 140 to 170° C.

In some embodiments, the method has a polyethylene terephthalate conversion of 72.5 to 100% based on the initial weight of polyethylene terephthalate at a temperature of greater than 170 to 210° C. The method has a selectivity of glycol ester of terephthalic acid of 82.5 to 99.9% at a temperature of greater than 170 to 210° C.

In some embodiments, the salt includes an alkali or alkaline earth metal cation and a carboxylate having 1 to 6 carbon atoms is present in an amount of 0.1 to 10 wt. % based on the total weight of polyethylene terephthalate. In some embodiments, the amine base is present in an amount of 0.1 to 10 wt. % based on a total weight of polyethylene terephthalate.

In some embodiments, the reaction mixture has a mole ratio of salt including an alkali or alkaline earth metal cation and a carboxylate having 1 to 6 carbon atoms to amine base of 2.5:1 to 1:2.5.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a bar graph depicting a comparison of polyethylene terephthalate (PET) degradation at 160° C. using various acetate salts (such as Na, K, Ca, Li acetate salts) and zinc acetate catalyst, according to certain embodiments.

FIG. 2 compares of PET degradation at 160° C. using various acetate salts (such as Na, K, Ca, Li acetate salts) versus formate salts (such as Na, K, Ca, Li formate salts), according to certain embodiments.

FIG. 3 compares PET degradation at 180° C. using various acetate salts (such as Na, Ca, Li acetate salts) and selected catalysts mixture containing a stoichiometric amount of 1,8-Diazabicyclo[5.4.0]undec-7-ene (DBU) base, according to certain embodiments.

FIG. 4 shows a plot of PET conversion and BHET selectivity vs time at 188° C. using lithium acetate and a stoichiometric amount of DBU base, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

As used herein, the term ‘cyclic amidine base’ refers to the cyclic organic compound that contains an amidine functional group, characterized by the presence of a nitrogen atom that is part of a ring structure, where at least one nitrogen atom is bonded to a carbon atom via a double bond and is also bonded to another nitrogen atom. These compounds exhibit basic properties and are utilized in various chemical applications, including catalysis and organic synthesis, due to their ability to stabilize reactive intermediates and facilitate chemical reactions.

As used herein, the term ‘amindine’ refers to the organic compound characterized by the functional group containing a carbon atom double-bonded to one nitrogen atom and single-bonded to another nitrogen atom (—C(═N—R)—N—). Amidines can exist as linear or cyclic structures and are recognized for their basic properties and utility in various chemical applications, including as intermediates in synthesis, catalysts, or as pharmaceutical agents.

According to a first aspect, the present disclosure relates to a method for the depolymerizing of polyethylene terephthalate (PET). As used herein, the term ‘depolymerization’ refers to the chemical process in which a polymer is broken down into its constituent monomers or smaller oligomeric units. The process involves the cleavage of the covalent bonds within the polymer chain, which can occur through various mechanisms such as thermal degradation, hydrolysis, or chemical reactions facilitated by catalysts. Depolymerization is significant in recycling and materials science, allowing for the recovery of monomers that can be reused in the synthesis of new polymers or other chemical products. Depolymerization is different from simple degradation or biodegradation in that depolymerization results in useful chemical species that can act as monomers or be readily converted into reactive monomers for polymerization.

In some embodiments, the method includes heating a reaction mixture including polyethylene terephthalate to 140 to 210° C., preferably 150 to 170° C., preferably 160° C. The reaction mixture includes polyethylene terephthalate. In some embodiments, the polyethylene terephthalate is a homopolymer. That is, the polyethylene terephthalate exists as polymer chains which contain polyethylene terephthalate but do not contain another polymer. In some embodiments, the polyethylene terephthalate is a copolymer. A copolymer refers to a polymer in which a polymer chain or backbone contains two or more distinct types of monomers. A copolymer can be classified by the arrangement of such monomers. Examples of type of copolymers include, but are not limited to, random copolymers, block copolymers, alternating copolymers, gradient copolymers, graft copolymers, periodic copolymers, and aperiodic copolymers. In general, the polyethylene terephthalate may be part of a polymer blend. A polymer blend can refer to a polymer that contains more than one type of polymer chain. Such polymer chains can be homopolymers or copolymers. Examples of other polymers that can be included with the polyethylene terephthalate, e.g., as a copolymer or a polymer in a polymer blend, include, but are not limited polyethylene naphthalate (PEN), biodegradable polyesters such as polylactic acid (PLA) and polyglycolic acid (PGA), polybutylene succinate (PBS), polycaprolactone (PCL), polypropylene terephthalate (PPT), polyethylene succinate, polybutylene adipate terephthalate, polybutylene terephthalate, polyethylene, polystyrene, poly(ethylene oxide), polybutadiene, thermoplastic starch blends, and the like. In some embodiments, polymers that are not polyethylene terephthalate can be removed prior to carrying out other steps of the method.

In some embodiments, the reaction mixture further includes a salt including an alkali or alkaline earth metal cation. As used herein, the term ‘alkali or alkaline earth metal cation’ refers to the positively charged ion originating from the alkali metals or alkaline earth metals, where alkali metal cations include Li⁺, Na⁺, K⁺, Rb⁺, Cs⁺, and alkaline earth metal cations include Be²⁺, Mg²⁺, Ca²⁺, Sr²⁺, Ba²⁺, and Ra²⁺. Examples of suitable salts including an alkali or alkaline earth metal cation include, but are not limited to halides, nitrates, phosphates, sulphates, carbonates, acetates, formates, and the like.

In some embodiments, the salt further includes a carboxylate having 1 to 6 carbon atoms. As used herein, the term ‘carboxylate’ refers to the negatively charged ion (anion) derived from a carboxylic acid by the deprotonation of its carboxyl group (—COOH). In a carboxylate ion, the hydrogen atom of the carboxyl group is replaced by a metal cation or another positively charged species, resulting in the general formula RCOO⁻, where R represents a hydrocarbon chain or group. Examples of the carboxylates having 1 to 6 carbon atoms include formate (HCOO⁻), acetate (CH₃COO⁻), propionate (C₂H₅COO⁻), butyrate (C₃H₇COO⁻), valerate (C₄H₉COO⁻), and caproate (C₅H₁₁COO⁻). In some embodiments, the carboxylate is formate. In some embodiments, the carboxylate is acetate. In some embodiments, the molar ratio of the alkali or alkaline earth metal cation to the carboxylate having 1 to 6 carbon atoms to urea of 2.5:1 to 1:2.5, preferably 2.25:1 to 1:2.25, preferably 2:1 to 1:2, preferably 1.75:1 to 1:1.75, preferably 1:1.5 to 1.5:1, preferably 1.25:1 to 1:1.25, preferably 1.1:1 to 1:1.1, preferably 1:1.

In some embodiments, the salt is the alkali or alkaline earth metal acetate salt. Examples of the alkali or alkaline earth metal acetate salts may include sodium acetate (NaC₂H₃O₂), potassium acetate (KC₂H₃O₂), calcium acetate (Ca(C₂H₃O₂)₂), magnesium acetate (Mg(C₂H₃O₂)₂), lithium acetate (LiC₂H₃O₂), zinc acetate (CH₃COO)₂Zn and barium acetate (Ba(C₂H₃O₂)₂). In some embodiments, the salt is the alkali or alkaline earth metal formate salt. Examples of alkali or alkaline earth metal formate salts include sodium formate (NaCHO₂), potassium formate (KCHO₂), calcium formate (Ca(HCO₂)₂), magnesium formate (Mg(HCO₂)₂), lithium formate (LiCHO₂), and barium formate (Ba(HCO₂)₂).

In some embodiments, the salt including the alkali or alkaline earth metal cation and the carboxylate having 1 to 6 carbon atoms is present in an amount of 0.1 to 10 wt. %, based on the total weight of polyethylene terephthalate. For example, the salt including the alkali or alkaline earth metal cation and the carboxylate having 1 to 6 carbon atoms can be present at 0.1 wt. %, 0.25 wt. %, 0.5 wt. %, 0.75 wt. %, 1.0 wt. %, 1.25 wt. %, 1.5 wt. %, 1.75 wt. %, 2.0 wt. %, 2.25 wt. %, 2.5 wt. %, 2.75 wt. %, 3.0 wt. %, 3.25 wt. %, 3.5 wt. %, 3.75 wt. %, 4.0 wt. %, 4.25 wt. %, 4.5 wt. %, 4.75 wt. %, 5.0 wt. %, 5.25 wt. %, 5.5 wt. %, 5.75 wt. %, 6.0 wt. %, 6.25 wt. %, 6.5 wt. %, 6.75 wt. %, 7.0 wt. %, 7.25 wt. %, 7.5 wt. %, 7.75 wt. %, 8.0 wt. %, 8.25 wt. %, 8.5 wt. %, 8.75 wt. %, 9.0 wt. %, 9.25 wt. %, 9.5 wt. %, 9.75 wt. %, or 10 wt. %, based on the total weight of polyethylene terephthalate.

In some embodiments, the reaction mixture further includes urea. In some embodiments, the urea is present in an amount of 0.1 to 10 wt. %, based on the total weight of polyethylene terephthalate. For example, the urea can be present at 0.1 wt. %, 0.25 wt. %, 0.5 wt. %, 0.75 wt. %, 1.0 wt. %, 1.25 wt. %, 1.5 wt. %, 1.75 wt. %, 2.0 wt. %, 2.25 wt. %, 2.5 wt. %, 2.75 wt. %, 3.0 wt. %, 3.25 wt. %, 3.5 wt. %, 3.75 wt. %, 4.0 wt. %, 4.25 wt. %, 4.5 wt. %, 4.75 wt. %, 5.0 wt. %, 5.25 wt. %, 5.5 wt. %, 5.75 wt. %, 6.0 wt. %, 6.25 wt. %, 6.5 wt. %, 6.75 wt. %, 7.0 wt. %, 7.25 wt. %, 7.5 wt. %, 7.75 wt. %, 8.0 wt. %, 8.25 wt. %, 8.5 wt. %, 8.75 wt. %, 9.0 wt. %, 9.25 wt. %, 9.5 wt. %, 9.75 wt. %, or 10 wt. %, based on the total weight of polyethylene terephthalate. In some embodiments, the reaction mixture is substantially free of urea.

In some embodiments, the reaction mixture further includes a glycol. As used herein, the term ‘glycol’ is a type of organic compound that contains two hydroxyl (—OH) groups attached to different carbon atoms. This structure categorizes glycols as diols, and they are typically colorless, odorless, and hygroscopic liquids. Examples of glycol include ethylene glycol (C₂H₆O₂), propylene glycol (C₃H₈O₂), butylene glycol (C₄H₁₀O₂), diethylene glycol (C₄H₁₀O₃), and triethylene glycol (C₆H₁₄O₄). In a preferred embodiment, the glycol is ethylene glycol.

In some embodiments, the reaction mixture further includes an amine base.

In some embodiments, the amine base is at least one selected from the group consisting of 1,8-Diazabicyclo(5.4.0)undec7-ene, 1,5-Diazabicyclo(4.3.0)non-5-ene,1,4-diazabicyclo[2.2.2]octane, 1,5,7-Triazabicyclo[4.4.0]dec-5-ene, N,N-Dimethylacetamide, Imidazole, 1-Methylimidazole, 2-Methylimidazole, Benzimidazole/Triphenylphosphine, 4-(Dimethylamino)pyridine, Triethylamine, Quinuclidine, 7-Methyl-1,5,7-triazabicyclo[4.4.0]dec-5-ene, 1,1,3,3-Tetramethylguanidine, 2-tert-Butylimino-2-diethylamino-1,3-dimethylperhydro-1,3,2-diazaphosphorine.

In some embodiments, the amine base is an amidine base. In some embodiments, the amidine base is at least one selected from the group consisting of 1,8-Diazabicyclo(5.4.0)undec7-ene, 1,5-Diazabicyclo(4.3.0)non-5-ene, 1,5,7-Triazabicyclo[4.4.0]dec-5-ene, 7-Methyl-1,5,7-triazabicyclo[4.4.0]dec-5-ene, 1,1,3,3-Tetramethylguanidine, 1,2-diazolidine, 1,3-diazepane, 1,3-dimethyl-2-imidazolidinone, 2-methyl-1,3-diazolidine, 1,2,3,4-tetrahydropyrimidine, 1,2-diazabicyclo[2.2.2]octane, 1,3-diazabicyclo[2.2.1]heptane, 1,4-diazabicyclo[3.3.0]octane, 1,3-dimethyl-2-imidazolidinone, and 2-methyl-1,3-diazolidine.

In some embodiments, the amine base is present in an amount of 0.1 to 10 wt. % based on the total weight of polyethylene terephthalate. For example, the amine base can be present at 0.1 wt. %, 0.25 wt. %, 0.5 wt. %, 0.75 wt. %, 1.0 wt. %, 1.25 wt. %, 1.5 wt. %, 1.75 wt. %, 2.0 wt. %, 2.25 wt. %, 2.5 wt. %, 2.75 wt. %, 3.0 wt. %, 3.25 wt. %, 3.5 wt. %, 3.75 wt. %, 4.0 wt. %, 4.25 wt. %, 4.5 wt. %, 4.75 wt. %, 5.0 wt. %, 5.25 wt. %, 5.5 wt. %, 5.75 wt. %, 6.0 wt. %, 6.25 wt. %, 6.5 wt. %, 6.75 wt. %, 7.0 wt. %, 7.25 wt. %, 7.5 wt. %, 7.75 wt. %, 8.0 wt. %, 8.25 wt. %, 8.5 wt. %, 8.75 wt. %, 9.0 wt. %, 9.25 wt. %, 9.5 wt. %, 9.75 wt. %, or 10 wt. %, based on the total weight of polyethylene terephthalate.

In some embodiments, the reaction mixture is substantially free of zinc.

In some embodiments, the heating causes depolymerization of polyethylene terephthalate, thereby forming a glycol ester of terephthalic acid including the glycol. In some embodiments, the glycol ester of terephthalic acid including the glycol is bis(2-hydroxyethyl) terephthalate.

In some embodiments, the method of the present disclosure has a polyethylene terephthalate conversion of 32 to 52.5%, based on the initial weight of polyethylene terephthalate at a temperature of 140 to 170° C. For example, the method can have a polyethylene terephthalate conversion of 32%, 32.5%, 33% 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, or 52.5%, based on the initial weight of polyethylene terephthalate at a temperature of 140 to 170° C. One of the factors that may influence the polyethylene terephthalate conversion is the choice of salt and/or the amine base in the reaction mixture. In some embodiments, the polyethylene terephthalate conversion is 32.3% when the salt is HCOONa. In some embodiments, the polyethylene terephthalate conversion is 33.6% when the salt is HCOOK. In some embodiments, the polyethylene terephthalate conversion is 34.4% when the salt is HCOOLi. In some embodiments, the polyethylene terephthalate conversion is 40.0% when the salt is (HCOO)₂Ca. In some embodiments, the polyethylene terephthalate conversion is 38.3% when the reaction mixture has CH₃COONa. In some embodiments, the polyethylene terephthalate conversion is 39.3% when the salt is CH₃COOK. In some embodiments, the polyethylene terephthalate conversion is 42.9% when the salt is (HCOO)₂Ca. In some embodiments, the polyethylene terephthalate conversion is 47.8% when the salt is CH₃COOLi.

In some embodiments, the polyethylene terephthalate conversion is 35.0% when the reaction mixture has CH₃COONa and 1,8-Diazabicyclo[5.4.0]undec-7-ene. In some embodiments, the polyethylene terephthalate conversion is 43.8% when the reaction mixture has CH₃COOLi and 1,8-Diazabicyclo[5.4.0]undec-7-ene. In some embodiments, the polyethylene terephthalate conversion is 46.2% when the reaction mixture has (HCOO)₂Ca and 1,8-Diazabicyclo[5.4.0]undec-7-ene. In some embodiments, the polyethylene terephthalate conversion is 39.1% when the reaction mixture has 1,8-Diazabicyclo[5.4.0]undec-7-ene.

In some embodiments, the method also has a selectivity of glycol ester of terephthalic acid of 82.5 to 99.9% at a temperature of 140 to 170° C., based on the initial weight of polyethylene terephthalate. For example, the method can have a selectivity of glycol ester of terephthalic acid of 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 99.9% at a temperature of 140 to 170° C., based on the initial weight of polyethylene terephthalate.

In some embodiments, the method has a polyethylene terephthalate conversion of 72.5 to 100% based on the initial weight of polyethylene terephthalate at a temperature of greater than 170 to 210° C. For example, the method can have a polyethylene terephthalate conversion of 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% based on the initial weight of polyethylene terephthalate at a temperature of greater than 170 to 210° C. In some embodiments, the polyethylene terephthalate conversion (e.g., when the heating is carried for 3 hours at 180° C. or 188° C.) is 100% when the reaction mixture has CH₃COOLi. In some embodiments, the polyethylene terephthalate conversion (e.g., when the heating is carried for 2 hours at 188° C.) is 79.1% when the reaction mixture has CH₃COOLi. In some embodiments, the polyethylene terephthalate conversion (e.g., when the heating is carried for 2.5 hours at 188° C.) is 93.3% when the reaction mixture has CH₃COOLi. In some embodiments, the method reaches a maximum conversion of polyethylene terephthalate in 2 to 4 hours, preferably 2.5 to 3.5 hours, preferably 3 hours.

In some embodiments, the method has a selectivity towards glycol ester of terephthalic acid of 82.5 to 99.9% at a temperature of greater than 170 to 210° C. For example, the method can have a selectivity of glycol ester of terephthalic acid of 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 99.9% at a temperature of greater than 170 to 210° C., based on the initial weight of polyethylene terephthalate. In some embodiments, the selectivity of glycol ester of terephthalic acid is 97% when the reaction mixture has CH₃COOLi. In some embodiments, the selectivity of glycol ester of terephthalic acid (e.g., when the heating is carried for 3 hours at 188° C.) is 89.6% when the reaction mixture has CH₃COOLi. In some embodiments, the selectivity of glycol ester of terephthalic acid (e.g., when the heating is carried for 2 hours at 188° C.) is 90.3% when the reaction mixture has CH₃COOLi. In some embodiments, the selectivity of glycol ester of terephthalic acid (e.g., when heating is carried for 2.5 hours at 188° C.) is 89.8% when the reaction mixture has CH₃COOLi.

### EXAMPLES

The following examples demonstrate an exemplary method of depolymerizing polyethylene terephthalate The examples are provided solely for illustration and are not to be construed as limitations of the present disclosure, as many variations thereof are possible without departing from the spirit and scope of the present disclosure.

For each experiment PET (0.5 g) was weighed out into a small Schlenk tube. Catalysts (1.0-4.0 wt. % equiv. each compared to PET) were weighed separately and added to the flask along with a magnetic stirrer. Ethylene Glycol (EG) (5 mL) was added using a syringe through the septum. The Schlenk tube was then dipped into an oil bath heated at desired temperature and allowed to stir for desired time. Once the reaction was complete, the Schlenk tubes were removed and an aliquot (0.1 mL) of reaction mixture was taken out using a syringe while hot and transferred to a vial. To this, DMSO-d⁶ (0.4 mL) was added while the sample was still hot, and this mixture was then transferred to an NMR tube and analyzed by 1H NMR using N-methyl pyrrolidone (NMP) as internal standard. Comparison of the integrals of the singlet from the methyl group of NMP δ=2.69 ppm and the singlet from the aromatic hydrogens from the depolymerization product (BHET) δ=8.12 ppm allowed calculation of the amount of BHET formed. The mixture was washed with water to remove excess EG and isolated remaining catalyst and the solid using a Buchner funnel. The solid BHET was then dried overnight in a vacuum oven to get the final weight.

PET conversion is defined by equation 1:

$${C}_{PET} = \frac{{W}_{0} - {W}_{1}}{{W}_{0}} \times 100 \% \qquad ( 1 )$$

where C_{PET} represents the conversion of PET, W₀ represents the initial weight of PET, and W₁ represents the weight of un-depolymerized PET.

The selectivity of BHET monomer is defined by equation 2:

$${S}_{BHET} = \frac{{n}_{BHET}}{{n}_{BHET ( Theo . )}} \times 100 \% \qquad ( 2 )$$

where S_{BHET} represents the selectivity of BHET, n_{BHET} represents moles of specific BHET products, and n_{BHET(Theo.)} represents the theoretical yield of BHET based on the number of moles of depolymerized PET units.

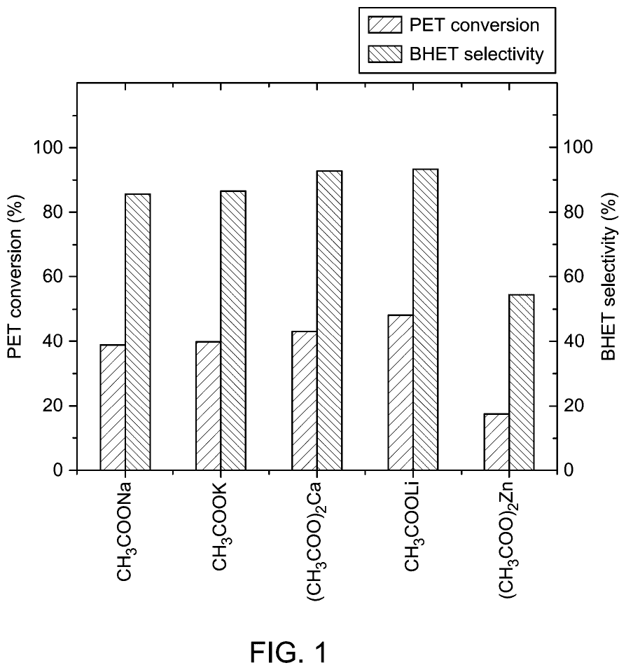

Catalytic activity was investigated by employing various readily available alkali/alkaline earth metal acetate salts at 160° C. (Table 1, entries 1~4 and FIG. 1). After a reaction duration of 3 hours, a moderate PET conversion was attained for all catalysts, with the highest (approximately 48%) observed for the lithium acetate catalyst (entry 4). Under the same conditions, zinc acetate catalysts exhibited significantly lower activity (entry 5). Importantly, in comparison to Zn acetate catalysts, all other newly investigated catalyst systems also demonstrated substantially higher BHET selectivity, typically ranging between 85% and 93%.

The impact of incorporating Lewis bases, namely urea and DBU, along with selected metal salts (Table 1, entries 6-11) was investigated. It was generally observed that DBU, when combined with metal salts, exhibited superior performance in terms of PET conversion. Although the BHET selectivity remained somewhat consistent. Under identical conditions, the zinc acetate/base pair once again displayed relatively lower PET conversion (Table 1, entries 12 and 13). Although a significant enhancement in BHET selectivity was observed for the Zn(CH₃COO)₂/DBU system, which could be attributed to the influence of DBU, this is supported by a catalytic run using pure DBU as a catalyst (entry 15), demonstrating a high BHET selectivity of approximately 83%.

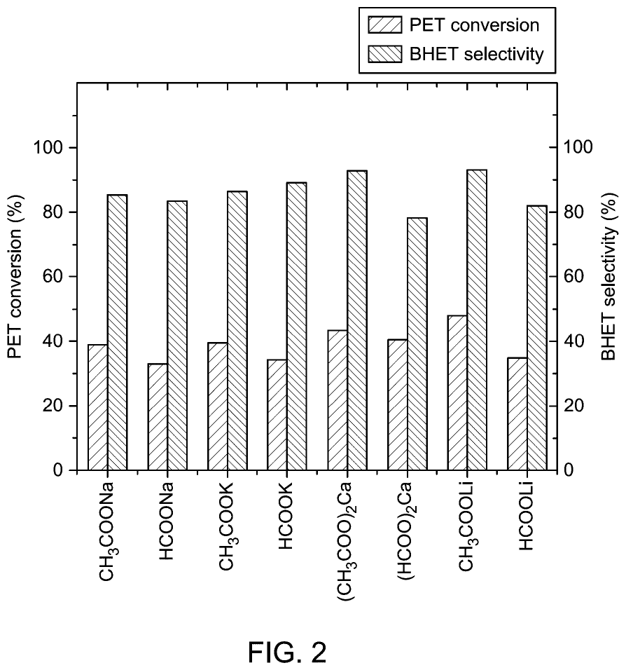

Subsequently, PET depolymerization using CF₃COONa and PhCOONa as catalysts was assessed. The results in entries 16 and 17 indicate that, compared to sodium acetate, these catalysts exhibit lower performance in terms of both PET conversion and BHET selectivity. To investigate the impact of carbon substitution, the formate salts of the targeted metal catalysts were examined. The data summarized in entries 18-21 (Table 1) and FIG. 2 indicates that these formate salts are relatively less active compared to the acetate salts.

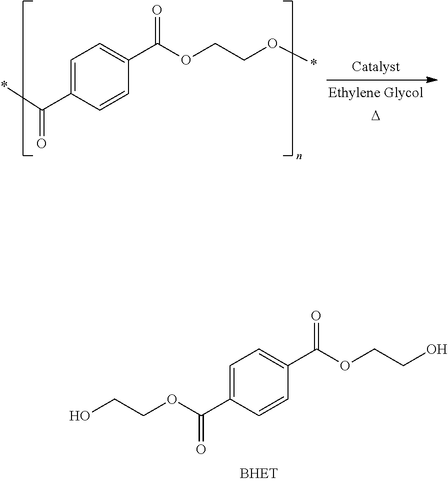

TABLE 1
PET depolymerization using various catalysts at 160° C. All
reactions were carried out using standard Schlenk (50 ml) and
heated at 160° C. for 3 h, with 0.50 g of PET, 5 mL ethylene
glycol and 4 wt. % catalyst; DBU = 1,8-Diazabicyclo[5.4.0]undec-7-ene.

| | | | | |
|---|---|---|---|---|
| | | Catalyst | PET | Yield of |
| | | loading | conversion | BHET |
| Entry | Catalyst | (wt. %) | (%) | (%) |

Alkali/alkaline metal acetate-based catalysts

| | | | | |
|---|---|---|---|---|
| 1 | CH₃COONa | 4 | 38.3 | 85.5 |
| 2 | CH₃COOK | 4 | 39.3 | 86.4 |
| 3 | (CH₃COO)₂Ca | 4 | 42.9 | 92.7 |
| 4 | CH₃COOLi | 4 | 47.8 | 93.1 |
| 5 | (CH₃COO)₂Zn | 4 | 16.9 | 54.1 |
| 6 | CH₃COONa:Urea | (2:2) | 33.9 | 88.3 |
| 7 | CH₃COONa + DBU | (2:2) | 35 | 85 |
| 8 | CH₃COOLi:Urea | (2:2) | 38.6 | 91 |
| 9 | CH₃COOLi:DBU | (2:2) | 43.8 | 92.2 |
| 10 | (CH₃COO)₂Ca:Urea | (2:2) | 39 | 88.1 |
| 11 | (CH₃COO)₂Ca:DBU | (2:2) | 46.2 | 90.7 |
| 12 | (CH₃COO)₂Zn:Urea | (2:2) | 11.1 | 52.3 |
| 13 | (CH₃COO)₂Zn:DBU | (2:2) | 20.3 | 73.7 |
| 14 | Urea | 4 | 17.5 | 56 |
| 15 | DBU | 4 | 39.1 | 82.8 |
| 16 | CF₃COONa | 4 | 19.9 | 72.8 |
| 17 | PhCOONa | 4 | 25.6 | 77.5 |

Alkali/alkaline metal-formate based catalysts

| | | | | |
|---|---|---|---|---|
| 18 | HCOONa | 4 | 32.3 | 83.3 |
| 19 | HCOOK | 4 | 33.6 | 89.2 |
| 20 | (HCOO)₂Ca | 4 | 40 | 78.2 |
| 21 | HCOOLi | 4 | 34.4 | 82 |

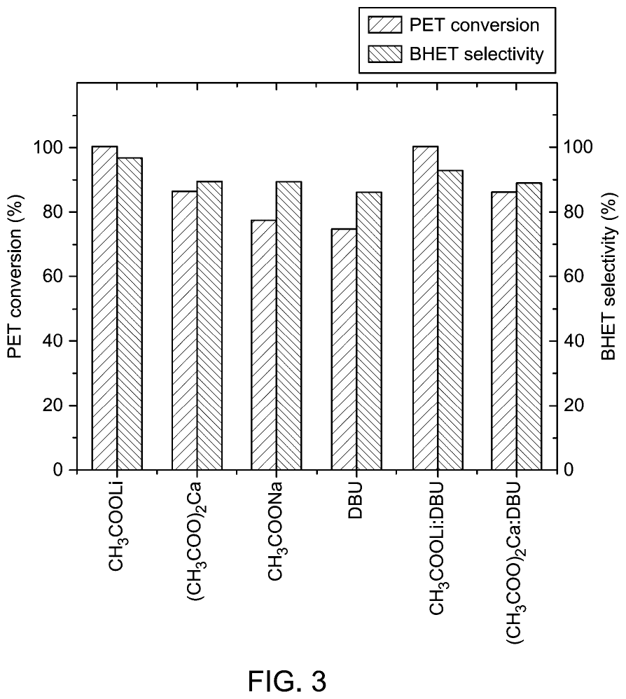

Catalytic runs were performed using selected catalyst systems at 180° C. in order to enhance the PET conversion efficiency while reducing the catalyst loading by half. The data presented in Table 2 and FIG. 3 indicate that Li salt outperforms other by giving 100% PET conversion with outstanding BHET selectivity (97%). A similar activity was again observed when CH₃COOLi/DBU (1:1) was employed as catalyst although the BHET selectivity was slightly lower (entry 5, Table 2). Under similar reaction condition, calcium acetate in presence or absence of DBU performed reasonably well by giving a PET conversion of about 86% and BHET selectivity 89%. The sodium acetate gave slightly weaker relative performance (entry 3, Table 2).

TABLE 2
PET depolymerization using various catalysts at 180° C. All
reactions were carried out using standard Schlenk (50 ml) and
heated at 180° C. for 3 h, with 0.50 g of PET, 5 mL ethylene
glycol and 2 wt. % catalyst; DBU = 1,8-Diazabicyclo[5.4.0]undec-7-ene.

| | | Catalyst | PET | Yield of |
|---|---|---|---|---|
| | | loading | conversion | BHET |
| Entry | Catalyst | (wt. %) | (%) | (%) |
| 1 | CH₃COOLi | 2 | 100 | 97 |
| 2 | (CH₃COO)₂Ca | 2 | 86.2 | 89.2 |
| 3 | CH₃COONa | 2 | 77.1 | 89 |
| 4 | DBU | 2 | 74.2 | 85.6 |
| 5 | CH₃COOLi:DBU | (1:1) | 100 | 92.5 |
| 6 | (CH₃COO)₂Ca:DBU | (1:1) | 85.9 | 88.8 |

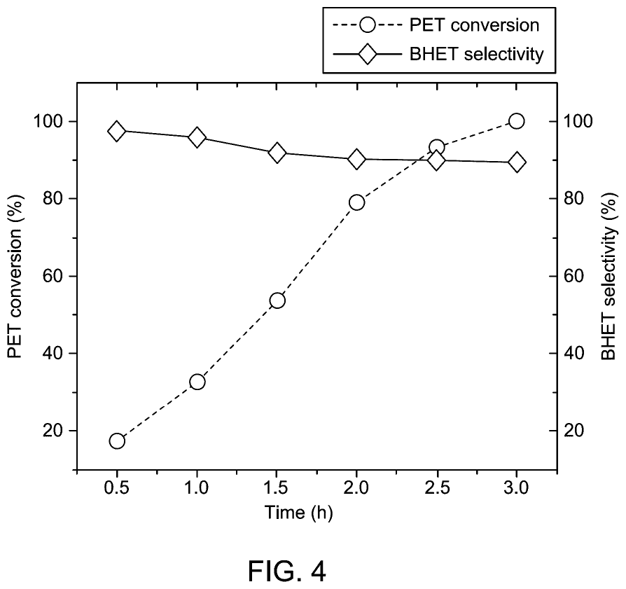

Following the assessment of the catalytic efficacy of lithium acetate salt, catalyst loading and reaction temperature were evaluated. The corresponding data and results are presented in FIG. 4 and Table 3. At 188° C. The lithium acetate catalyst was found to attain a 93% PET conversion within 2.5 hours and complete conversion within 3 hours. Notably, a clear linear correlation between PET conversion and time was evident up to 2.5 hours. Exceptionally high BHET selectivity was consistently maintained throughout the experiments.

TABLE 3
A time dependent PET depolymerization study using lithium
acetate as catalyst at 188° C. All reactions were carried
out using standard Schlenk (50 ml) and heated at 188°
C., 0.50 g of PET, 5 mL ethylene glycol and 1 wt. % catalyst.

| Entry | Time (h) | PET conversion (%) | Yield of BHET (%) |
|---|---|---|---|
| 1 | 0.5 | 17.3 | 97.5 |
| 2 | 1 | 32.7 | 96 |
| 3 | 1.5 | 53.8 | 92.1 |
| 4 | 2 | 79.1 | 90.3 |
| 5 | 2.5 | 93.3 | 89.8 |
| 6 | 3 | 100 | 89.6 |

TABLE 4
Time-dependent PET depolymerization study at 188° C. using
various catalysts. All reactions were conducted in standard
50 mL Schlenk flasks and heated at 188° C., using 0.50
g of PET, 5 mL of ethylene glycol, and 0.076 mmol of catalyst.

| | | Time | PET conversion | Yield of BHET |
|---|---|---|---|---|
| Entry | Catalyst | (h) | (%) | (%) |
| 1 | CH₃COOLi | 0.5 | 17.3 | 16.9 |
| 2 | | 1 | 32.7 | 31.4 |
| 3 | | 1.5 | 53.8 | 49.5 |
| 4 | | 2 | 79.1 | 71.4 |
| 5 | | 2.5 | 93.3 | 83.8 |
| 6 | | 3 | 99.9 | 89.6 |
| 7 | CH₃COO)₂Zn•2H₂O | 0.5 | 15.0 | 12.9 |
| 8 | | 1 | 27.1 | 22.9 |
| 9 | | 1.5 | 46.0 | 37.8 |
| 10 | | 2 | 58.2 | 47.7 |
| 11 | | 2.5 | 69.3 | 55.4 |
| 12 | | 3 | 91.0 | 71.8 |
| 13 | DBU | 0.5 | 17.1 | 14.5 |
| 14 | | 1 | 31.0 | 26.1 |
| 15 | | 1.5 | 52.9 | 44.6 |
| 16 | | 2 | 68.9 | 57.5 |
| 17 | | 2.5 | 90.1 | 75.2 |
| 18 | | 3 | 97.3 | 77.4 |
| 19 | (CH₃COO)₂Ca | 0.5 | 28.5 | 24.51 |
| 20 | | 1 | 42 | 31 |
| 21 | | 1.5 | 62 | 48.3 |
| 22 | | 2 | 79 | 70.8 |
| 23 | | 2.5 | 95 | 82 |
| 24 | | 3 | 100 | 86 |

TABLE 5
Comparative initial reaction rates of various catalysts at 188°
C. All reactions were conducted in standard 50 mL Schlenk flasks
and heated at 188° C., using 0.50 g (2.604 mmol) of PET,
0.076 mmol of catalyst, and 5 mL of ethylene glycol.

| Catalyst | Time | PET conversion (%) | TOF (h⁻¹) |
|---|---|---|---|
| CH₃COOLi | 0.5 | 17.3 | 11.9 |
| (CH₃COO)₂Zn•2H₂O | 0.5 | 15 | 10.3 |
| DBU | 0.5 | 17.1 | 11.5 |
| (CH₃COO)₂Ca | 0.5 | 28.53 | 19.6 |

Turnover Frequency (TOF) calculated using formula (3) below:

$$TOF = \frac{m {ol}_{PET converted}}{mo {l}_{Cat . used} \times time ( h )} \qquad ( 3 )$$

TABLE 6
Comparative initial reaction rates of various catalysts
including Na- and K-acetate at 188° C. All reactions
were conducted in standard 50 mL Schlenk flasks and heated
at 188° C., using 0.50 g (2.604 mmol) of PET, 0.076
mmol of catalyst, and 5 mL of ethylene glycol.

| | Time | PET conversion | TOF |
|---|---|---|---|
| Catalyst | (h) | (%) | (h⁻¹) |
| CH₃COOLi | 0.5 | 17.3 | 11.9 |
| (CH₃COO)₂Zn•2H₂O | | 15 | 10.3 |
| (CH₃COO)Ca | | 28.5 | 19.6 |
| CH₃COONa | | 9.5 | 6.4 |
| CH₃COOK | | 13.8 | 9.5 |

The present method may be advantageous for utilizing environmentally friendly metal catalysts such as lithium, sodium, or calcium acetate/formate. Unlike traditional methods that rely on heavy metal salts, which pose toxicity and environmental concerns, the present method circumvents these drawbacks by employing more benign alternatives. Further, the present method may be advantageous in that it shows enhanced degradation efficiency of PET and significantly improved selectivity for BHET. The method may allow for complete PET conversion at lower temperatures and with minimal catalyst loading. The present method may offer a cost-effective and sustainable solution for recycling PET waste while addressing environmental and health issues associated with heavy metal catalysts.

Numerous modifications and variations of the present disclosure are possible in light of the above teachings. It is therefore to be understood that within the scope of the appended claims, the invention may be practiced otherwise than as specifically described herein.

1. A method of depolymerizing polyethylene terephthalate, the method comprising
heating to 140 to 210° C. a reaction mixture comprising
polyethylene terephthalate,
a salt comprising an alkali or alkaline earth metal cation and a carboxylate having 1 to 6 carbon atoms,
urea, and
a glycol, wherein
the depolymerizing forms a glycol ester of terephthalic acid comprising the glycol,
the urea is present in an amount of 0.1 to 10 wt. % based on a total weight of polyethylene terephthalate,
the salt comprising an alkali or alkaline earth metal cation and a carboxylate having 1 to 6 carbon atoms is present in an amount of 0.1 to 10 wt. % based on a total weight of polyethylene terephthalate, and
the reaction mixture further comprises an amine base.

2. The method of claim 1, wherein
the salt comprising an alkali or alkaline earth metal cation and a carboxylate having 1 to 6 carbon atoms is at least one selected from the group consisting of lithium acetate, lithium formate, sodium acetate, sodium formate, potassium acetate, potassium formate, calcium acetate, calcium formate, magnesium acetate, and magnesium formate.

3. The method of claim 1, wherein
the glycol is ethylene glycol.

4. The method of claim 3, wherein
the glycol ester of terephthalic acid comprising the glycol is bis(2-hydroxyethyl) terephthalate.

5. The method of claim 1, wherein
the amine base is at least one selected from the group consisting of 1,8-Diazabicyclo(5.4.0)undec7-ene, 1,5-Diazabicyclo(4.3.0)non-5-ene, 1,5,7-Triazabicyclo[4.4.0]dec-5-ene, 7-Methyl-1,5,7-triazabicyclo[4.4.0]dec-5-ene, 1,1,3,3-Tetramethylguanidine, 1,2-diazolidine, 1,3-diazepane, 1,3-dimethyl-2-imidazolidinone, 2-methyl-1,3-diazolidine, 1,2,3,4-tetrahydropyrimidine, 1,2-diazabicyclo[2.2.2]octane, 1,3-diazabicyclo[2.2.1]heptane, 1,4-diazabicyclo[3.3.0]octane, 1,3-dimethyl-2-imidazolidinone, and 2-methyl-1,3-diazolidine.

6. The method of claim 1, wherein
the amine base is present in an amount of 0.1 to 10 wt. % based on a total weight of polyethylene terephthalate.

7. The method of claim 1, wherein
the reaction mixture has a mole ratio of salt comprising an alkali or alkaline earth metal cation and a carboxylate having 1 to 6 carbon atoms to urea of 2.5:1 to 1:2.5.

8. The method of claim 1, having
a polyethylene terephthalate conversion of 32 to 52.5% based on an initial weight of polyethylene terephthalate at a temperature of 140 to 170° C.; and
a selectivity of glycol ester of terephthalic acid of 82.5 to 99.9% at a temperature of 140 to 170° C.

9. The method of claim 1, having
a polyethylene terephthalate conversion of 72.5 to 100% based on an initial weight of polyethylene terephthalate at a temperature of greater than 170 to 210° C.; and
a selectivity of glycol ester of terephthalic acid of 82.5 to 99.9% at a temperature of greater than 170 to 210° C.

10. The method of claim 1, wherein
the method reaches a maximum conversion of polyethylene terephthalate in 2 to 4 hours.

11. The method of claim 1, wherein
the reaction mixture is substantially free of zinc.

12. A method of depolymerizing polyethylene terephthalate, the method comprising
heating to 140 to 210° C. a reaction mixture comprising
polyethylene terephthalate,
a salt comprising an alkali or alkaline earth metal cation and a carboxylate having 1 to 6 carbon atoms,
an amine base, and
a glycol, wherein
the depolymerizing forms a glycol ester of terephthalic acid comprising the glycol; and
the reaction mixture is substantially free of urea.

13. The method of claim 12, wherein
the amine base is at least one selected from the group consisting of 1,8-Diazabicyclo(5.4.0)undec7-ene, 1,5-Diazabicyclo(4.3.0)non-5-ene, 1,5,7-Triazabicyclo[4.4.0]dec-5-ene, 7-Methyl-1,5,7-triazabicyclo[4.4.0]dec-5-ene, 1,1,3,3-Tetramethylguanidine, 1,2-diazolidine, 1,3-diazepane, 1,3-dimethyl-2-imidazolidinone, 2-methyl-1,3-diazolidine, 1,2,3,4-tetrahydropyrimidine, 1,2-diazabicyclo[2.2.2]octane, 1,3-diazabicyclo[2.2.1]heptane, 1,4-diazabicyclo[3.3.0]octane, 1,3-dimethyl-2-imidazolidinone, and 2-methyl-1,3-diazolidine.

14. The method of claim 12, having
a polyethylene terephthalate conversion of 32 to 52.5% based on an initial weight of polyethylene terephthalate at a temperature of 140 to 170° C.; and
a selectivity of glycol ester of terephthalic acid of 82.5 to 99.9% at a temperature of 140 to 170° C.

15. The method of claim 12, having
a polyethylene terephthalate conversion of 72.5 to 100% based on an initial weight of polyethylene terephthalate at a temperature of greater than 170 to 210° C.; and
a selectivity of glycol ester of terephthalic acid of 82.5 to 99.9% at a temperature of greater than 170 to 210° C.

16. The method of claim 12, wherein
the salt comprising an alkali or alkaline earth metal cation and a carboxylate having 1 to 6 carbon atoms is present in an amount of 0.1 to 10 wt. % based on a total weight of polyethylene terephthalate; and
the amine base is present in an amount of 0.1 to 10 wt. % based on a total weight of polyethylene terephthalate.

17. The method of claim 12, wherein
the reaction mixture has a mole ratio of salt comprising an alkali or alkaline earth metal cation and a carboxylate having 1 to 6 carbon atoms to amine base of 2.5:1 to 1:2.5.
